(19) United States
(12) Patent Application Publication
(10) Pub. No.: US 2022/0037020 A1
(43) Pub. Date: Feb. 3, 2022

(54) MODELING EXTERNAL EVENT EFFECTS UPON SYSTEM VARIABLES

(71) Applicant: International Business Machines Corporation, Armonk, NY (US)

(72) Inventors: Debarun Bhattacharjya, New York, NY (US); Tian Gao, Berkeley Heights, NJ (US); Nicholas Scott Mattei, New Orleans, LA (US); Karthikeyan Shanmugam, Elmsford, NY (US); Dharmashankar Subramanian, White Plains, NY (US); Kush Raj Varshney, Ossining, NY (US)

(21) Appl. No.: 16/942,842

(22) Filed: Jul. 30, 2020

(51) Int. Cl. G16H 50/20; G16H 50/70; G06N 20/20; G06N 7/00

(52) U.S. Cl. CPC G16H 50/20; G06N 7/005; G06N 20/20; G16H 50/70

(57) ABSTRACT

Analyzing complex systems by receiving labeled event data describing events occurring in association with a complex system, generating a first machine learning model according to the distribution of labeled event data, receiving state variable transition data describing state variable transitions occurring in association with a complex system, training a second machine learning model according to a combination of a distribution of state variable transitions and the first machine learning model, and using the second machine learning model to predict the effects of events upon state variables within the complex system according to new state variable transition and new labeled event data.

### STATEMENT REGARDING PRIOR DISCLOSURES BY THE INVENTOR OR A JOINT INVENTOR

The following disclosure(s) are submitted under 35 U.S.C. § 102(b)(1)(A):

### DISCLOSURE(S)

(1) Debarun Bhattacharjya, Karthikeyan Shanmugam, Tian Gao, Nicholas Mattei, Kush Varshney, Dharmashankar Subramanian. (2020). Event-Driven Continuous Time Bayesian Networks. In Thirty-Fourth AAAI Conference on Artificial Intelligence Thirty-Second Conference on Innovative Applications of Artificial Intelligence The Tenth Symposium on Educational Advances in Artificial Intelligence (pp. 3259-3266). Feb. 7-12, 2020, New York Hilton Midtown, New York, N.Y., USA. Published by AAAI Press, Palo Alto, Calif.

### BACKGROUND

The disclosure relates generally to modeling the effects of external events upon system variables. The disclosure relates particularly to modeling the effects of external events upon system state variables using an event-driven, continuous-time, Bayesian network (ECTBN).

Real-world situations often involve variables that interact with each other through complex, dynamic interdependencies. Variables of interest in a system may be modeled as state variables with values captured by a dynamic process at regular or irregular intervals rather than continuously. A continuous-time-Bayesian network (CTBN) may be used to model the joint trajectories of state variables having irregular state transitions. The CTBN models the variables as homogeneous Markov processes.

### SUMMARY

The following presents a summary to provide a basic understanding of one or more embodiments of the disclosure. This summary is not intended to identify key or critical elements or delineate any scope of the particular embodiments or any scope of the claims. Its sole purpose is to present concepts in a simplified form as a prelude to the more detailed description that is presented later. In one or more embodiments described herein, devices, systems, computer-implemented methods, apparatuses and/or computer program products enable the analysis of systems having complex interdependencies.

Aspects of the invention disclose methods, systems and computer readable media associated with analyzing complex systems by receiving labeled event data describing events occurring in association with a complex system, generating a first machine learning model according to the distribution of labeled event data, receiving state variable transition data describing state variable transitions occurring in association with a complex system, generating a second machine learning model according to a combination of a distribution of state variable transitions and the first machine learning model, and using the second machine learning model to predict the effects of events upon state variables within the complex system according to new state variable transition and new labeled event data.

### BRIEF DESCRIPTION OF THE DRAWINGS

Through the more detailed description of some embodiments of the present disclosure in the accompanying drawings, the above and other objects, features and advantages of the present disclosure will become more apparent, wherein the same reference generally refers to the same components in the embodiments of the present disclosure.

FIG. 1 provides a graphical example of a complex system subject to modelling by embodiments of the invention.

FIG. 2 provides a graphical example of a complex system modeled by an embodiment of the invention.

FIG. 3 provides a graphical example of a complex system modelled by an embodiment of the invention.

FIG. 4 provides a schematic illustration of a computing environment, according to an embodiment of the invention.

FIG. 5 provides a flowchart depicting an operational sequence, according to an embodiment of the invention.

### DETAILED DESCRIPTION

Some embodiments will be described in more detail with reference to the accompanying drawings, in which the embodiments of the present disclosure have been illustrated. However, the present disclosure can be implemented in various manners, and thus should not be construed to be limited to the embodiments disclosed herein.

In an embodiment, one or more components of the system can employ hardware and/or software to solve problems that are highly technical in nature (e.g., training a first machine learning model according to historic event sequence data, training a second machine learning model according to state variable changes over time and the first machine learning model, using the second machine learning models to predict the effects of current of future events upon variable states, etc.). These solutions are not abstract and cannot be performed as a set of mental acts by a human due to the processing capabilities needed to facilitate complex system modeling, for example. Further, some of the processes performed may be performed by a specialized computer for carrying out defined tasks related to system modeling. For example, a specialized computer can be employed to carry out tasks related to modeling complex systems, or the like.

CTBNs offer a mechanism to model state variable dynamics for an isolated system. Such networks may not be suitable for modeling systems wherein external events influence the evolution of the system state variables over time. Disclosed systems and methods provide ways to model complex systems where various types of external events may also influence the evolution of the system state variables. The models include joint dynamics involving both event occurrences, modeled as a multivariate point process, and state variables, modeled as Markov processes.

Such complex systems may include, without being limiting, health-related system including the influence of events such as insulin intake, meals and physical activity upon a diabetic patient's blood glucose level and mental well-being; stock prices for a set of companies in an industry affected by natural events such as disasters or political events such as trade deals; the impact of social services, such as counseling sessions and classes, on a person's level of education, employment, and well-being.

Event datasets include sequences of labels on a timeline. Each time stamped event label indicates the type of event and its relative position upon an event timeline. For example, labeled time stamps of medication, exercise, and meals would indicate events that could be relevant for a patient's health outcomes. To capture the influence of events on state variables, disclosed embodiments utilize event-driven continuous time Bayesian networks (ECTBNs)—where, in addition to state variables driving transitions of other state variables over a time duration, previous, current and future time-stamped events could influence the time to transition as well as the probability of transition of state variables.

Including events in the scope of the model requires a fundamental extension to CTBNs and cannot be reduced to an expanded CTBN with proxy state variables for events. This is because the intensity function that determines time to next transition in a CTBN only depends on the current configuration of parent state variables; it does not depend on when the configuration of these state variables attained their current configuration. However, when event sequences influence the intensity functions of state transitions, their previous times of occurrence could matter, making the influence non-Markov because it does not only depend on the current state.

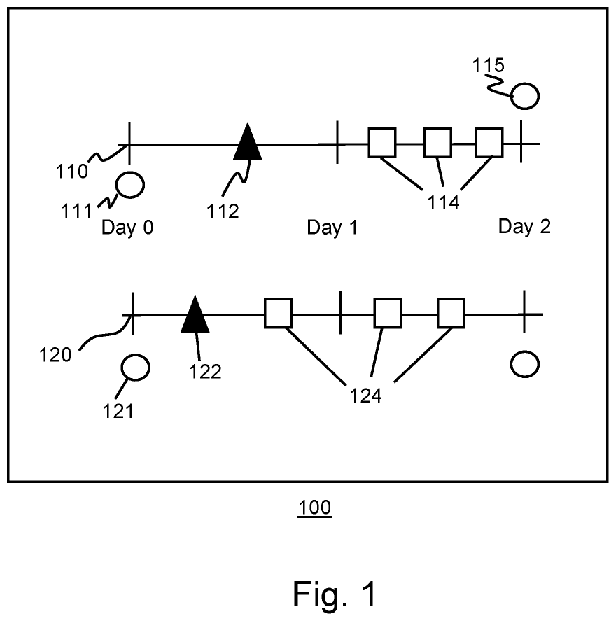

As an example, consider the case where the frequency of meals in the recent history affects transitions of a patient's blood sugar levels. This is illustrated in schematic 100 of FIG. 1 where a blood sugar state variable with two states, low and high, is influenced by exercise and meal events over two separate two-day sequence timelines 110 and 120. As shown in the Figure, blood sugar transitions from low 111, to high 115, over the two-day span of timeline 110 after the occurrence of an exercise event 112 on day 1, and after three meals 114 on day 2. As shown on timeline 120, blood sugar does not transition from low 121 to high, after an exercise event 122 on day one as well as a meal 124 on day 1 and two additional meals 124, on day 2. Even if the events were modeled as state variables and the sequences of events were tracked with memory, the intensity function would be unable to capture the notion that only the number of meals within a certain time window influences the blood sugar level.

For purposes of this disclosure, a set of discrete state variables χ={Xᵢ}ᵢ₌₁^{l}. Val(Xᵢ) represents the domain of variable Xᵢ. The states of these variables are known over the span of a time duration, at all times between initial time t₀=0 to the end time T. Data about each variable is of the form of state transitions, D_{X}_{ᵢ}=(tₖ, xₖ)ₖ₌₀^{N}^{ᵢ} where the state at time to is the initial state and xₖ₊₁≠xₖ∀k,xₖ∈Val(Xᵢ). Data for all state variables taken together is denoted D_{X}= [symbol] D_{X}.

The method also utilizes data about events occurring over a time duration, D_{ε}=(tₖ, eₖ)ₖ₌₁^{N}^{_{E}}, where tₖ are time stamps and eₖ belong to an event label set ε={Eⱼ}ⱼ₌₁^{J}. All the data taken together is D=D_{X}U∪D_{ε}. The method uses h(⋅) to denote historical occurrences of events. h_{B}(t)={(tₖ,eₖ)∈D_{B}: tₖ<t} represents the history of events in the set B⊂ε until time t.

In an embodiment having a set of state variables and a set of labeled historic event data, disclosed methods create an ECTBN for the system. The ECTBN includes a directed (possibly cyclic) graph G where U_{E}⊂E are parents of event label E and U_{X}⊂{ [symbol] \X} ∪ε are parents of state variable X∈ [symbol] . The method decomposes the latter into two sets: state variable parents U_{X(χ)} ⊆ [symbol] \X and parents that are event labels U_{X(ε)}⊆ε.

The method considers an initial distribution P⁰_{X} of state variables, and conditional intensity matrices for every X∈ [symbol] , Q_{(X|u_(X(χ),), h(UX(ε))} (t)), which model state transitions. The matrices depend upon the current state u_{X(X)} of the parents U_{X(X)} at time t and history of labels in U_{X(ε)} till time t, denoted h_{U}_{_{X(E)}}(t). A matrix Q(⋅) is equivalent to considering waiting times q_{x|uX(X),h} U_{X(∈)} (t) in state X=x before transitioning to some other state x′≠x, as well as the probabilities of transitioning from state x to state x′ at time t, θ_{xx′|uX(X),h} U_{X(ε)} (t). The method also considers conditional intensity rates for every event label E∈ε, λ_{E|h}U_{E}(t), which model event arrivals. The history of event labels in parent set U_{E} at time t is denoted h_{U}_{_{E}}(t).

In an embodiment, the learning for the combined ECTBN model includes a recency assumption relating to the effect of events upon state variables—according to the assumption, recent events matter more than older ones. For a set [symbol] of time windows for every edge from event label E directed into state variable X in graph G, each denoted w_{(E,X)}. the rates and probabilities associated with state variable transitions depend only on whether a parent event label E∈U_{X(ε)} occurred at least once in some recent time window w_{(E,X)}. Given data D about state transitions and event occurrences and a complete set of hyper-parameters of windows for every edge from E to X, [symbol] , the learning phase seeks the ECTBN graph G and model parameters.

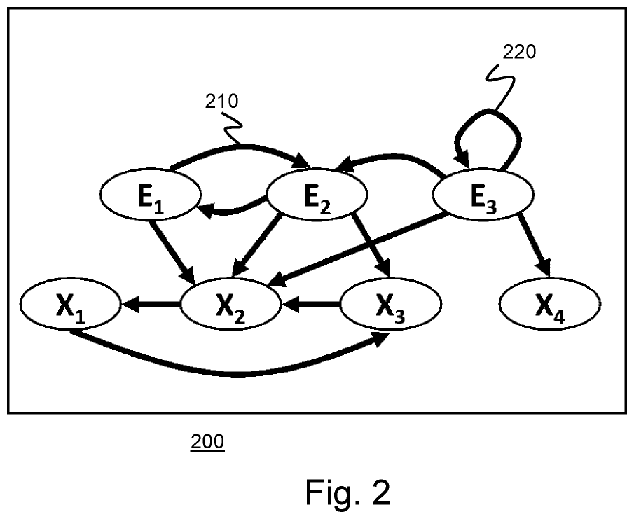

Schematic 200 of FIG. 2 shows an illustrative ECTBN graph for four state variables X₁, X₂, X₃, and X₄, as well as 3 events E₁, E₂, and E₃. Note that there may be cycles 210, shown for E1 and E₂ even self-loops 220 as shown for E₃, because its occurrence rate could depend on its own history. State variables Xᵢ could have event labels as parents but not vice versa. In this embodiment, the method studies situations where events could probabilistically influence the uncertainties in a system but not the other way around.

For a set W of time windows for every edge from event label E directed into state variable X in graph G, each denoted w_{(E,X)}, the method assumes that the rates and probabilities associated with state variable transitions depend only on whether a parent event label E∈U_{X(E)} occurred at least once in some recent time window w_{(E,X)}.

This is the recency or proximal assumption: recent events matter more than older ones. This assumption simplifies parent conditions to be binary for each parent. Specifically, if u_{X(E)} denotes a vector of indicators, one each for whether an event label in U_{X(E)} occurs or not, then the recency assumption simplifies the dependence of q(⋅) and θ(⋅) as:

q_{x|uX(} [symbol] _{),hUX(ε)(t)}=q_{x|uX(} [symbol] _{),uX(ε)}; θ_{xx0|uX(} [symbol] _{),hUX(ε)(t)}=θ_{xx0|uX(} [symbol] _{),uX(ε)}

The number of parameters can now be ascertained for any state variable. As an example, for the ECTBN in FIG. 2, if state variable X₃ has 3 values in its domain Val(X3), then X₂ has 2³*3=24 parental conditions (u_{X(χ)},u_{X(ε)}) since it has 3 event labels as parents, U_{X2(ε)}={E1,E2,E3}, along with 1 state variable parent, [symbol] ={X₃}.

This method extends easily to a case where state variable parameters are a piece-wise constant function of the history of events. In an embodiment, the method uses a general class of functions to model dependence on event histories instead of a function involving only the most recent time window. The piece-wise constant model is general enough to approximate arbitrary histories. In this example, the method considers only recent windows to avoid the notation from getting unwieldy. The method may utilize the recency assumption due to the nature of real-world causal influences, and to avoid overfitting.

In an embodiment, given data D about state transitions and event occurrences and a complete set of hyperparameters of windows for every edge from E to X, W^{c}, the method finds the ECTBN graph G and model parameters. In this embodiment, the method focuses upon learning state variable parameters and their dependence on events.

The likelihood of observed data from D can be factorized as the combination of the likelihood of the state variable transition and the likelihood of an event arrival. The method seeks an optimal graph combining state transition and event arrival likelihoods.

For a system where Q={q,Θ} represent the collection of q_{(⋅)} and θ_{(⋅)} parameters that model the state variable transitions. Similarly, A represents the collection of λ_{(⋅)} parameters for the arrival sequence of events. The likelihood of observed data factorizes according to the graph G, by:

L(D|Q,Λ)=[Π_{X∈X}L(D_{X}|Q,D_{U}_{_{X(X)}},D_{U}_{_{X(E)}})][Π_{E∈ε}L(D_{E}|Λ,D_{U}_{_{E}})]

The data likelihood for a state variable X is a function of the parameters for waiting times and probabilities of transitions. In the general case, these depend on the history of events. For brevity in the following equation, h(t) represents the joint historical condition u_{X(χ)},h_{U}_{_{X(ε)}}(t). The method factors likelihood as:

$$L ( {D}_{X} \vert Q , {D}_{{U}_{X ( X )}} , {D}_{{U}_{X ( E )}} ) = {\prod }_{( {t}_{k} , {x}_{k} ) ∈ {D}_{X}} {θ}_{{x}_{k} {x}_{k + 1} \vert h ( {t}_{k + 1} )} {\prod }_{( {t}_{k} , {x}_{k} ) ∈ D} {q}_{{x}_{k} \vert h ( {t}_{k + 1} )} * {e}^{( - {\int }_{{t}_{k}}^{{t}_{k + 1}} {q}_{{x}_{k} \vert h ( t )} dt )} \qquad$$

The data likelihood for arrivals of an event label E depends on the event arrival rates:

$$L ( {D}_{X} \vert {QD}_{E} ) = [ {Π}_{x ∈ Val ( X ) , X ∈ X} {Π}_{u} {q}_{x \vert u}^{M [ x \vert u ]} {e}^{- T [ x \vert u ] {q}_{x \vert u}} ] [ {Π}_{x \neq {x}^{′} ∈ Val ( X ) , X ∈ X} {Π}_{u} {θ}_{{xx}^{′} \vert u}^{M [ x , {x}^{′} \vert u ]} ]$$

The above expression is quite general and covers most reasonable multivariate point processes In this example, the method focuses solely on learning state variable parameters Q given a graph G, omitting details about learning event arrival process parameters Λ, though any of a number of models could be deployed for this purpose.

In an embodiment, u represents a vector that takes values in Val(u_{X(χ)})×Val(u_{X(ε)}) for any X∈ [symbol] :

$$L ( {D}_{X} \vert Q , {D}_{E} ) = [ {\prod }_{x ∈ Val ( X ) , X ∈ X} {\prod }_{u} {q}_{x \vert u}^{M [ x \vert u ]} {e}^{- T [ x \vert u ] {q}_{x \vert u}} ] [ {\prod }_{x \neq x ′ ∈ Val ( X ) , X ∈ X} {\prod }_{u} {θ}_{xx ′ \vert u}^{M [ x , {x}^{′} \vert u ]} ]$$

The summary statistics for X are defined as: M[x,x′|u]: the number of times the variable transitions from state x to state x′ and the condition u is true at those times, i.e., when u_{X(χ)} and u_{X(ε)} take values in u; M[x|u]: the number of times the variable transitions from state x and the condition u is true at those times, i.e., when u_{X(χ)} and u_{X(ε)} take values in u; T[x|u]: the total amount of time where the variable is in state x and the condition u is true at those times, i.e., when u_{X(χ)} and u_{X(ε)} take values in u.

The maximum likelihood estimates for parameters q and Θ given the structure G are:

$${\hat{q}}_{x \vert u} = \frac{M [ x \vert u ]}{T [ x \vert u ]} ; {\hat{θ}}_{x x ′ \vert u} = \frac{M [ x , {x}^{′} \vert u ]}{M [ x \vert u ]}$$

In an embodiment, ECTBN methods reveal relationships between events and state variables. Determining a true graph of the complex system reveals information about events that change a current variable state to a new variable state.

The method uses G*=max_{G} s(G,D), to find the optimal graph. s(G,D) is a scoring function that measures the fit between any graph G and data D. The Bayesian Information Criterion (BIC) score, adapted to ECTBNs, defined for state variable X as:

$$BIC ( X ) = log L ( {D}_{𝓍} ) - [ \frac{log \vert D \vert}{2} Dim ( Q ( X ) ) ] ,$$

where |D| is the size of the data. Dim(Q(X)) is the dimensionality of the parameters for X, which in our case is the number of independent parameters in q and Θ that are associated with X:

Dim(Q(X))=|Val(X)|²*2^{|UX(E)|}*Q_{Z∈UX(X)}|Val(Z)|.

The method decomposes learning the true or optimal graph into learning individual optimal sub-graphs and then combining them to form the global optimal graph. Using a sub-graph learning approach finds the optimal parent set of each state variable X with a hill climbing search. At each iteration, the method chooses the highest scoring graph among the set of graphs consisting of the current graph and all graphs that are one operation away from the current graphs. The operations include adding an edge and deleting an edge. The search for the parents for each node continues until there is no improvement in scores.

Unlike a CTBN, an ECTBN is able to incorporate historical dependencies of event arrivals. In an embodiment, using the recency assumption, i.e., rates and state transitions depend on u_{X(E)} that denotes whether the individual events E∈U_{E(X)} occurred in time window w_{(E,X)} or not. As a test of the method, 3 models were generated, each with 5 state variables and 5 event label variables. The models differed in the structural relations among the state variables: they included a chain, a star (naive Bayes like structure), and a cycle. The method utilizes synthetic test data where the ground truth ECTBN graph and parameters are known.

The chain model has a chain graph structure among state variables: X₁→X₂→X₃→X₄→X₅. Each state variable has 3 random event label parents. The star model has a naive Bayes graphical structure among variables: X₁→X₂, X₁→X₃, X₁→X₄, and X₁→X₅. Again, each state variable has 3 random event label parents.

The cycle model forms a circle with its state variables: X₁→X₂→X₃→X₄→X₅→X₁. In this model, each state variable has 2 random event label parents. In all three models, each of 5 event labels can have 2 to 4 other event labels as parents, but with no state variables as parents as per the ECTBN assumptions.

For all three models, each state variable has three states. State variable parameters q_{(⋅)} and θ_{(⋅)} were generated randomly from a uniform distribution between 0.1 to ⅓ and a Dirichlet distribution with hyperparameter α=(1,1) respectively. Event traces were generated from a proximal graphical event model (PGEM) with windows ranging from 10 to 30 and rate of 0.5. Other parameters follow default values. Windows from event parents to state variables were set to 15. For each model, 10 datasets were generated over time period T=10K that include PGEM generated event traces as well as state variable transitions which are unique to an ECTBN.

Table 1 shows graph structure recovery results of the ECTBN learner for all variables' parents (both state variables and event labels) in these 3 synthetic models. The average precision and recall of each variable's parent's function as the performance measure for the learned graph structure against the ground truth. The data indicates that the precision is excellent for all models, but the recall varies and is model dependent. Precision refers to the relevance of returned results, whereas recall refers to the proper classification of returned results. There is perfect recall for the cycle model—all results are properly classified. Structure recovery is in general a challenging task and while this is also the case for ECTBNs. The data further shows that the learner has very low false positive rates, indicated by the high precision, along with reasonable false negative rates, indicated by the recall values.

TABLE 1

| Model | Precision | Recall |
|---|---|---|
| Chain | 97% | 47.4% |
| Star | 84.6% | 57.9% |
| Cycle | 100% | 100% |

In one example, the ECTBN model was used to study the effect of a set of services (events) on an individual's life outcome areas (state variables) in an integrated social services initiative. For the example, the data was associated with approximately 1400 clients who each had more than 15 total social services interactions out of a total of over 2900 total clients. The example considered 6 outcome areas that are tracked through the data: education, employment, financial education, transportation, anxiety, and depression. These are dimensions of an individual's progress in attaining a self-sustainable way out of poverty. Each of these six outcome areas has between three and six levels (states). The example considered 11 types of provided services, which were treated as events: 6 of them relate to group classes/sessions and 5 relate to one-on-one sessions. The services include group industrial training, group classes on education, employment, financial education, transportation and wellness, as well as one-on-one sessions on employment, wellness, and financial education.

The following learning procedure was applied to the data and conducted separately for each state variable (outcome area) X. First, a hyper-parameter setting was configured for windows in W^{c} associated with incoming edges into X by uniformly randomly choosing a window from the list {15,30,60,90,180} days for each event label. This procedure was repeated 100 times to build various window hyperparameter configurations. Using 5-fold cross validation, the method determined the optimal hyper-parameter setting by maximizing the average BIC score across folds. Finally, this optimal hyper-parameter setting was used to learn the optimal graph and parameters for X using all the training data.

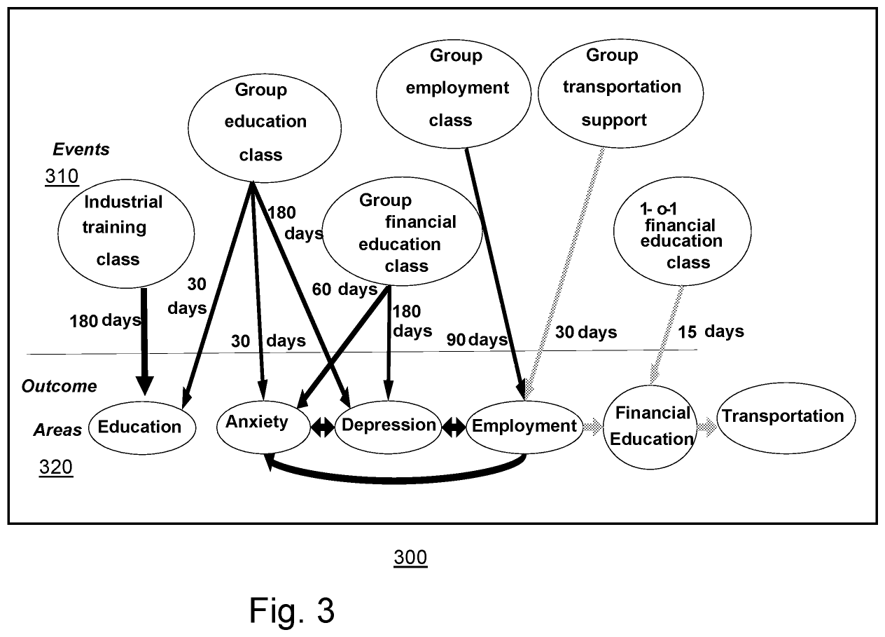

ECTBN graph 300 of FIG. 3 presents the learned graphical structure and windows for the data. learned using a slightly reduced weight for the penalty term in the BIC score, due to limited data. ECTBN 300 includes the relationships between respective Events 310 and Outcome areas 320. There are several interesting results that can be gleaned from the graph 300, potentially affecting the way social services are offered. First, group education classes have a direct and lasting effect on the Anxiety and Depression outcome areas, as do group financial education classes. Industrial training classes have a longer duration of effect (180 days) on the Education outcome area than the other group education classes (30 days). One-on-one financial education classes have more impact on the Financial Education outcome area than group financial education classes. Employment has a direct effect on Anxiety, Depression, and Financial Education. The data shows that Anxiety, Depression, and Employment are critical, reinforcing the importance of a holistic approach to case management.

A study was conducted to better identify influential events that affect transitions from a particular outcome area level to the next level. This was done by creating additional state variables to track when the level of an outcome area increased; each new state variable has three states—the current level (not the maximum level), the next higher level and some other level of the outcome area under consideration. An ECTBN was learned for each new state variable while considering other outcome areas and events.

Table 3 summarizes the ECTBN event parents for three outcome areas determined from this transition analysis, enabling identification of local effects that were not evident previously. Selecting a few of these additional insights: (1) core education classes are important for transitions at lower levels of education whereas industrial training is important for transitions at higher levels; (2) the impact of group employment classes is particularly felt on low to mid-levels of employment transitions; and (3) group financial education classes affect lower level transitions whereas the one-on-one classes are influential throughout the progression. For this analysis, all windows were set to 180 days during learning.

TABLE 2

| Outcome | | | | | | |
|---|---|---|---|---|---|---|
| Area | Level 1 | Level 2 | Level 3 | Level 4 | Level 5 | Level 6 |
| Education | group edu | group edu | group edu | indus. | indus. | N/A |
| | class | class | class; | training | training | |
| | | | indus. | | | |
| | | | training | | | |
| Employment | group emp | group emp | group emp | — | — | N/A |
| | class; | class; | class; | | | |
| | group | group | group | | | |
| | transp. | transp. | transp. | | | |
| | class | class | class | | | |
| Financial | 1-on-1 fin- | 1-on-1 fin- | 1-on-1 fin- | 1-on-1 fin- | N/A | N/A |
| | ed; | ed; | ed | ed | | |
| Education | group fin- | group fin- | | | | |
| | ed | ed | | | | |

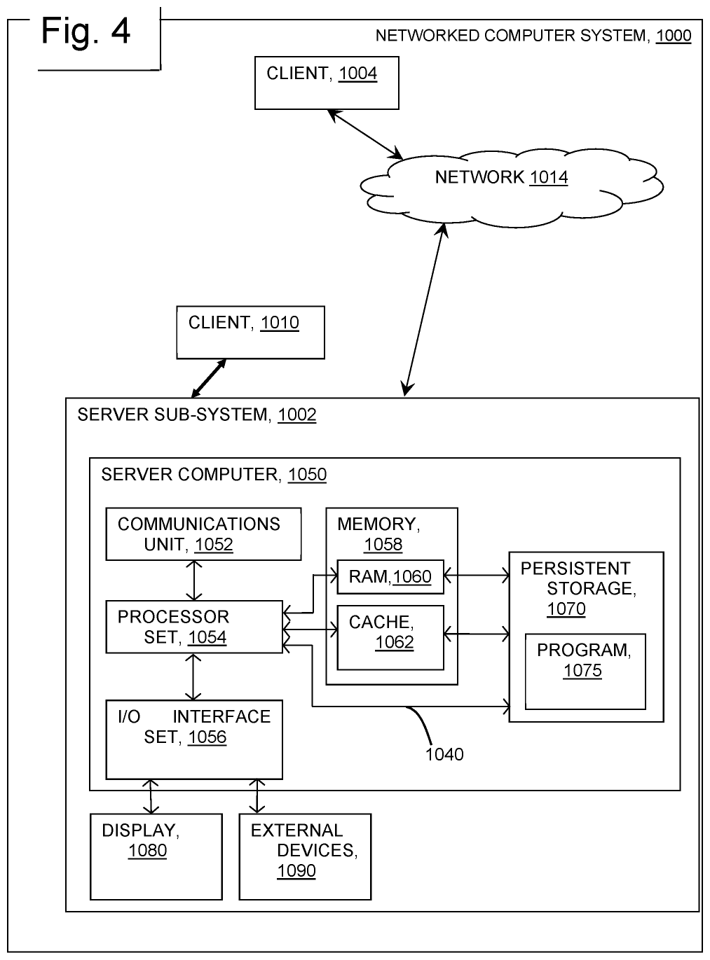

FIG. 4 provides a schematic illustration of exemplary network resources associated with practicing the disclosed inventions. The inventions may be practiced in the processors of any of the disclosed elements which process an instruction stream. As shown in the figure, a networked Client device 1010 connects wirelessly to server sub-system 1002. Client device 1004 connects wirelessly to server sub-system 1002 via network 1014. Client devices 1004 and 1010 comprise application program (not shown) together with sufficient computing resource (processor, memory, network communications hardware) to execute the program. In an embodiment, client devices form portions of an overall ECTBN computing environment and enable the gathering of system event and state variable transition data, as well as enabling user access to ECTBN models and results. As shown in FIG. 4, server sub-system 1002 comprises a server computer 1050. FIG. 4 depicts a block diagram of components of server computer 1050 within a networked computer system 1000, in accordance with an embodiment of the present invention. It should be appreciated that FIG. 4 provides only an illustration of one implementation and does not imply any limitations with regard to the environments in which different embodiments can be implemented. Many modifications to the depicted environment can be made.

Server computer 1050 can include processor(s) 1054, memory 1058, persistent storage 1070, communications unit 1052, input/output (I/O) interface(s) 1056 and communications fabric 1040. Communications fabric 1040 provides communications between cache 1062, memory 1058, persistent storage 1070, communications unit 1052, and input/output (I/O) interface(s) 1056. Communications fabric 1040 can be implemented with any architecture designed for passing data and/or control information between processors (such as microprocessors, communications and network processors, etc.), system memory, peripheral devices, and any other hardware components within a system. For example, communications fabric 1040 can be implemented with one or more buses.

Memory 1058 and persistent storage 1070 are computer readable storage media. In this embodiment, memory 1058 includes random access memory (RAM) 1060. In general, memory 1058 can include any suitable volatile or non-volatile computer readable storage media. Cache 1062 is a fast memory that enhances the performance of processor(s) 1054 by holding recently accessed data, and data near recently accessed data, from memory 1058.

Program instructions and data used to practice embodiments of the present invention, e.g., the systems analysis program 1075, are stored in persistent storage 1070 for execution and/or access by one or more of the respective processor(s) 1054 of server computer 1050 via cache 1062. In this embodiment, persistent storage 1070 includes a magnetic hard disk drive. Alternatively, or in addition to a magnetic hard disk drive, persistent storage 1070 can include a solid-state hard drive, a semiconductor storage device, a read-only memory (ROM), an erasable programmable read-only memory (EPROM), a flash memory, or any other computer readable storage media that is capable of storing program instructions or digital information.

The media used by persistent storage 1070 may also be removable. For example, a removable hard drive may be used for persistent storage 1070. Other examples include optical and magnetic disks, thumb drives, and smart cards that are inserted into a drive for transfer onto another computer readable storage medium that is also part of persistent storage 1070.

Communications unit 1052, in these examples, provides for communications with other data processing systems or devices, including resources of client computing devices 1004, and 1010. In these examples, communications unit 1052 includes one or more network interface cards. Communications unit 1052 may provide communications through the use of either or both physical and wireless communications links. Software distribution programs, and other programs and data used for implementation of the present invention, may be downloaded to persistent storage 1070 of server computer 1050 through communications unit 1052.

I/O interface(s) 1056 allows for input and output of data with other devices that may be connected to server computer 1050. For example, I/O interface(s) 1056 may provide a connection to external device(s) 1090 such as a keyboard, a keypad, a touch screen, a microphone, a digital camera, and/or some other suitable input device. External device(s) 1090 can also include portable computer readable storage media such as, for example, thumb drives, portable optical or magnetic disks, and memory cards. Software and data used to practice embodiments of the present invention, e.g., systems analysis program 1075 on server computer 1050, can be stored on such portable computer readable storage media and can be loaded onto persistent storage 1070 via I/O interface(s) 1056. I/O interface(s) 156 also connect to a display 1080.

Display 1080 provides a mechanism to display data to a user and may be, for example, a computer monitor. Display 1080 can also function as a touch screen, such as a display of a tablet computer.

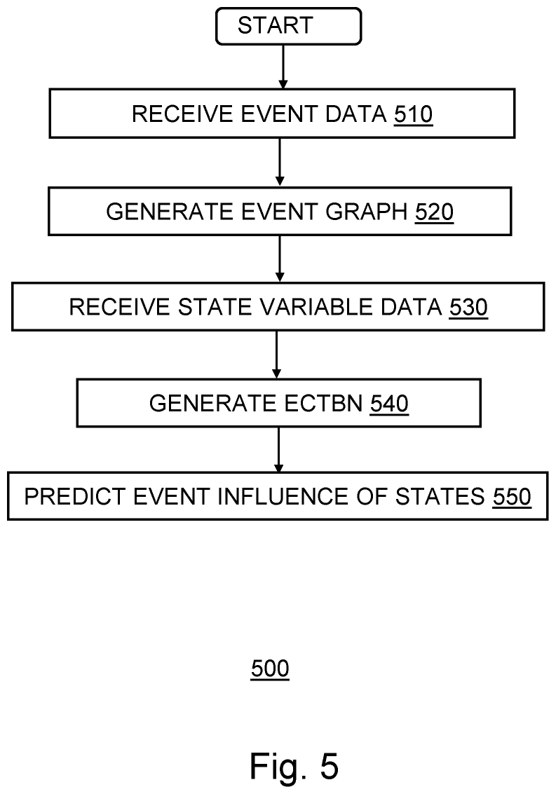

FIG. 5 provides a flowchart 500, illustrating exemplary activities associated with the practice of the disclosure. After program start, at block 510, the method receives a dataset including labeled event data over time for a complex system, at block 520, the method creates a first machine learning model such as event graph, from the labeled event data for the system. The first machine learning model relates the likelihood associated with the occurrences of the historic events. At block 530, the method receives state variable transition data for the complex system. At block 540, the method generates and trains a second machine learning model, as an example, the method generates an ECTBN model from the historic state variable transition data for the system and the first machine learning model, e.g., the event graph. The ECTBN depicts the relationships between state variables and events. At block 550, the method uses the ECTBN to predict the effect of events upon state variable transitions using current or forecast event and or state variable transition data for the system. In an embodiment, the method utilizes the ECTBN to predict the events needed (event injections) to achieve desired state variable transitions using new, real-time state transition data and labeled event data.

The present invention may be a system, a method, and/or a computer program product at any possible technical detail level of integration. The invention may be beneficially practiced in any system, single or parallel, which processes an instruction stream. The computer program product may include a computer readable storage medium (or media) having computer readable program instructions thereon for causing a processor to carry out aspects of the present invention.

The computer readable storage medium can be a tangible device that can retain and store instructions for use by an instruction execution device. The computer readable storage medium may be, for example, but is not limited to, an electronic storage device, a magnetic storage device, an optical storage device, an electromagnetic storage device, a semiconductor storage device, or any suitable combination of the foregoing. A non-exhaustive list of more specific examples of the computer readable storage medium includes the following: a portable computer diskette, a hard disk, a random access memory (RAM), a read-only memory (ROM), an erasable programmable read-only memory (EPROM or Flash memory), a static random access memory (SRAM), a portable compact disc read-only memory (CD-ROM), a digital versatile disk (DVD), a memory stick, a floppy disk, a mechanically encoded device such as punch-cards or raised structures in a groove having instructions recorded thereon, and any suitable combination of the foregoing. A computer readable storage medium, or computer readable storage device, as used herein, is not to be construed as being transitory signals per se, such as radio waves or other freely propagating electromagnetic waves, electromagnetic waves propagating through a waveguide or other transmission media (e.g., light pulses passing through a fiber-optic cable), or electrical signals transmitted through a wire.

Computer readable program instructions described herein can be downloaded to respective computing/processing devices from a computer readable storage medium or to an external computer or external storage device via a network, for example, the Internet, a local area network, a wide area network and/or a wireless network. The network may comprise copper transmission cables, optical transmission fibers, wireless transmission, routers, firewalls, switches, gateway computers and/or edge servers. A network adapter card or network interface in each computing/processing device receives computer readable program instructions from the network and forwards the computer readable program instructions for storage in a computer readable storage medium within the respective computing/processing device.

Computer readable program instructions for carrying out operations of the present invention may be assembler instructions, instruction-set-architecture (ISA) instructions, machine instructions, machine dependent instructions, microcode, firmware instructions, state-setting data, configuration data for integrated circuitry, or either source code or object code written in any combination of one or more programming languages, including an object oriented programming language such as Smalltalk, C++, or the like, and procedural programming languages, such as the “C” programming language or similar programming languages. The computer readable program instructions may execute entirely on the user's computer, partly on the user's computer, as a stand-alone software package, partly on the user's computer and partly on a remote computer or entirely on the remote computer or server. In the latter scenario, the remote computer may be connected to the user's computer through any type of network, including a local area network (LAN) or a wide area network (WAN), or the connection may be made to an external computer (for example, through the Internet using an Internet Service Provider). In some embodiments, electronic circuitry including, for example, programmable logic circuitry, field-programmable gate arrays (FPGA), or programmable logic arrays (PLA) may execute the computer readable program instructions by utilizing state information of the computer readable program instructions to personalize the electronic circuitry, in order to perform aspects of the present invention.

Aspects of the present invention are described herein with reference to flowchart illustrations and/or block diagrams of methods, apparatus (systems), and computer program products according to embodiments of the invention. It will be understood that each block of the flowchart illustrations and/or block diagrams, and combinations of blocks in the flowchart illustrations and/or block diagrams, can be implemented by computer readable program instructions.

These computer readable program instructions may be provided to a processor of a general purpose computer, special purpose computer, or other programmable data processing apparatus to produce a machine, such that the instructions, which execute via the processor of the computer or other programmable data processing apparatus, create means for implementing the functions/acts specified in the flowchart and/or block diagram block or blocks. These computer readable program instructions may also be stored in a computer readable storage medium that can direct a computer, a programmable data processing apparatus, and/or other devices to function in a particular manner, such that the computer readable storage medium having instructions stored collectively therein comprises an article of manufacture including instructions which implement aspects of the function/act specified in the flowchart and/or block diagram block or blocks.

The computer readable program instructions may also be loaded onto a computer, other programmable data processing apparatus, or other device to cause a series of operational steps to be performed on the computer, other programmable apparatus or other device to produce a computer implemented process, such that the instructions which execute on the computer, other programmable apparatus, or other device implement the functions/acts specified in the flowchart and/or block diagram block or blocks.

The flowchart and block diagrams in the Figures illustrate the architecture, functionality, and operation of possible implementations of systems, methods, and computer program products according to various embodiments of the present invention. In this regard, each block in the flowchart or block diagrams may represent a module, segment, or portion of instructions, which comprises one or more executable instructions for implementing the specified logical function(s). In some alternative implementations, the functions noted in the blocks may occur out of the order noted in the Figures. For example, two blocks shown in succession may, in fact, be executed substantially concurrently, or the blocks may sometimes be executed in the reverse order, depending upon the functionality involved. It will also be noted that each block of the block diagrams and/or flowchart illustration, and combinations of blocks in the block diagrams and/or flowchart illustration, can be implemented by special purpose hardware-based systems that perform the specified functions or acts or carry out combinations of special purpose hardware and computer instructions.

References in the specification to “one embodiment”, “an embodiment”, “an example embodiment”, etc., indicate that the embodiment described may include a particular feature, structure, or characteristic, but every embodiment may not necessarily include the particular feature, structure, or characteristic. Moreover, such phrases are not necessarily referring to the same embodiment. Further, when a particular feature, structure, or characteristic is described in connection with an embodiment, it is submitted that it is within the knowledge of one skilled in the art to affect such feature, structure, or characteristic in connection with other embodiments whether or not explicitly described.

The terminology used herein is for the purpose of describing particular embodiments only and is not intended to be limiting of the invention. As used herein, the singular forms “a,” “an,” and “the” are intended to include the plural forms as well, unless the context clearly indicates otherwise. It will be further understood that the terms “comprises” and/or “comprising,” when used in this specification, specify the presence of stated features, integers, steps, operations, elements, and/or components, but do not preclude the presence or addition of one or more other features, integers, steps, operations, elements, components, and/or groups thereof.

The descriptions of the various embodiments of the present invention have been presented for purposes of illustration but are not intended to be exhaustive or limited to the embodiments disclosed. Many modifications and variations will be apparent to those of ordinary skill in the art without departing from the scope and spirit of the invention. The terminology used herein was chosen to best explain the principles of the embodiment, the practical application or technical improvement over technologies found in the marketplace, or to enable others of ordinary skill in the art to understand the embodiments disclosed herein.

What is claimed is:

1. A computer implemented method for analyzing complex systems, the method comprising:
receiving, by one or more computer processors, labeled event data describing events occurring in association with a complex system;
generating, by the one or more computer processors, a first machine learning model according to a distribution of the labeled event data;
receiving, by the one or more computer processors, state variable transition data describing state transitions occurring in association with the complex system;
generating, by the one or more computer processors, a second machine learning model according to a combination of a distribution of state transition data and the first machine learning model; and
using, by the one or more computer processors, the second machine learning model to predict effects of events upon state variables within the complex system according to new state variable transition data and new labeled event data.

2. The computer implemented method according to claim 1, wherein the first machine learning model comprises a graphical model considering labeled event history during a time duration.

3. The computer implemented method according to claim 1, wherein the second machine learning model comprises a Bayesian network model considering state variable transitions during a time duration.

4. The computer implemented method according to claim 1, wherein the complex system comprises a health-related system.

5. The computer implemented method according to claim 1, wherein the state variable transition data and the labeled event data are time stamped.

6. The computer implemented method according to claim 1, further comprising computing, by the one or more computer processors, event injections to achieve desired state variable transitions.

7. The computer implemented method according to claim 1, further comprising predicting, by the one or more computer processors, state variable transitions according to real-time labeled event data.

8. A computer program product for analyzing complex system, the computer program product comprising one or more computer readable storage devices and collectively stored program instructions on the one or more computer readable storage devices, the stored program instructions comprising:
program instructions to receive labeled event data describing events occurring in association with a complex system;
program instructions to generate a first machine learning model according to a distribution of the labeled event data;
program instructions to receive state variable transition data describing events occurring in association with the complex system;
program instructions to generate a second machine learning model according to a combination of a distribution of state variable transition data, and the first machine learning model; and
program instructions to use the second machine learning model to predict effects of events upon state variables within the complex system according to new state variable transition and new labeled event data.

9. The computer program product according to claim 8, wherein the first machine learning model comprises a graphical model considering labeled event history during ae time duration.

10. The computer program product according to claim 8, wherein the second machine learning model comprises a Bayesian network model considering state variable transitions during a time duration.

11. The computer program product according to claim 8, wherein the complex system comprises a health-related system.

12. The computer program product according to claim 8, wherein the state variable transition and the labeled event data are time stamped.

13. The computer program product according to claim 8, the stored program instructions further comprising program instructions to compute event injections to achieve desired state variable transitions.

14. The computer program product according to claim 8, the stored program instructions further comprising program instructions to predict state variable transitions according to real-time labeled event data.

15. A computer system for analyzing complex systems, the computer system comprising:
one or more computer processors;
one or more computer readable storage devices; and
stored program instructions on the one or more computer readable storage devices for execution by the one or more computer processors, the stored program instructions comprising:
program instructions to receive labeled event data describing events occurring in association with a complex system;
program instructions to generate a first machine learning model according to a distribution of the labeled event data;
program instructions to receive state variable transition data describing state variable transitions occurring in association with the complex system;
program instructions to generate a second machine learning model according to a combination of a distribution of the state variable transitions and the first machine learning model; and
program instructions to use the second machine learning model to predict effects of events upon state variables within the complex system according to new state variable transition data and new labeled event data.

16. The computer system according to claim 15, wherein the first machine learning model comprises a graphical model considering labeled event data history during a time duration.

17. The computer system according to claim 15, wherein the second machine learning model comprises a Bayesian network model considering state variable transitions during a time duration.

18. The computer system according to claim 15, wherein the state variable transition data and the labeled event data are time stamped.

19. The computer system according to claim 15, the stored program instructions further comprising program instructions to compute event injections to achieve desired state variable transitions.

20. The computer system according to claim 15, the stored program instructions further comprising program instructions to predict state variable transitions according to real-time labeled event data.
